(19) United States
(12) Patent Application Publication
(10) Pub. No.: US 2019/0274961 A1
(43) Pub. Date: Sep. 12, 2019

(54) CONTROLLED RELEASE TABLET BASED ON POLYVINYL ALCOHOL AND ITS MANUFACTURING

(71) Applicant: Merck Patent GmbH, Darmstadt (DE)

(72) Inventors: Mengyao ZHENG, Darmstadt (DE); Nicole DI GALLO, Bensheim (DE); Anja-Nadine KNUETTEL, Mannheim (DE)

(73) Assignee: Merck Patent GmbH, Darmstadt (DE)

(21) Appl. No.: 16/347,604

(22) PCT Filed: Nov. 6, 2017
(86) PCT No.: PCT/EP2017/078267

§ 371 Date: May 6, 2019

(30) Foreign Application Priority Data
Nov. 7, 2016 (EP) 16197610.5

(51) Int. Cl. A61K 9/20; A61K 9/14; A61K 47/32

(52) U.S. Cl. CPC A61K 9/2095; A61K 31/405; A61K 47/32; A61K 9/141

(57) ABSTRACT

The present invention relates to an improved powdered extrudate based on polyvinyl alcohol (PVA), which can be used for the production of pharmaceutical products, and due to its improved properties, can be better directly compressed into tablets. Furthermore, this invention refers to pharmaceutical tablets composition comprising extruded polyvinyl alcohol as carrier matrix, which is suitable to improve the solubility of API within a controlled release (instant or sustained) kinetic.

The present invention relates to powdered polyvinyl alcohol having improved properties as a polymer matrix in pharmaceutical formulations comprising active ingredients, especially in compressed tablets forming amorphous solid dispersions with poorly soluble APIs. Furthermore, the invention relates to such compositions with controlled release and to processes for preparing these preparations and to their use.

### TECHNICAL FIELD

Here the term “solid dispersion” is understood to mean a dispersion in a polymer matrix of the amorphous active ingredient. Preferably, the amorphous active ingredient is molecularly dispersely distributed in the polymer matrix. In this case, the solid dispersion is a solid solution.

Solid dispersions are defined as being a dispersion of one or more active ingredients in an inert solid matrix and can broadly be classified as those containing a drug substance in the crystalline state or in the amorphous state [Chiou W. L., Riegelman S. Pharmaceutical applications of Solid dispersion systems; J. Pharm Sci. 1971, 60 (9), 1281-1301].

In order to achieve a more consistent dosage rate of the active ingredient in pharmaceutical formulations, it is useful when the active ingredient is present as a homogeneous solid dispersion or as solution in a carrier. Solid dispersions containing pharmaceutical active ingredients in the crystalline state provide dissolution enhancement by simply decreasing surface tension, reducing agglomeration, and improving wettability of the active substance [Sinswat P., et al.; Stabilizer choice for rapid dissolving high potency itraconazole particles formed by evaporative precipitation into aqueous solution; Int. J. of Pharmaceutics, (2005) 302; 113-124]. While crystalline systems are more thermodynamically stable than their amorphous counterparts, the crystalline structure must be interrupted during the dissolution process, requiring energy, in order to produce a solid dispersion. The term “solid dispersion containing an active ingredient” means, that a drug is dissolved at the molecular level in a matrix or carrier. This state is known as amorphous solid solution and can result in a significant increase in dissolution rate and extent of supersaturation [DiNunzio J. C. et al. III Amorphous compositions using concentration enhancing polymers for improved bioavailability of itraconazole; Molecular Pharmaceutics (2008); 5(6):968-980].

While these systems have several advantages, physical instability can be problematic due to molecular mobility and due to the tendency of the drug to recrystallize. Polymeric carriers with high glass transition temperatures seem to be well suited to stabilize these systems by limiting molecular mobility.

As such, solid dispersions can be created by a number of methods, including, but not limited to, spray-drying, melt extrusion, and thermokinetic compounding.

Although hot melt extrusion (HME), a fusion processing technique, has been used in the food and plastics industry for more than a century, it has only recently gained acceptance in the pharmaceutical industry for the preparation of formulations comprising active ingredients processed by extrusion. And now, HME has been introduced as pharmaceutical manufacturing technology and has become a well-known process with benefits like continuous and effective processing, limited number of process steps, solvent free process etc.

During hot melt extrusion the active ingredients are mixed with and embedded in excipients, such as polymers and plasticizers. Furthermore, drug substances are exposed to elevated temperatures for a period of time. Although a variety of factors can affect the residence time distribution of an extruded substance, these times typically fall within the 1- to 2-min range [Breitenbach J., Melt extrusion: from process to drug delivery technology. Eur J Pharm Biopharm. (2002), 54, 107-117].

Therefore, as carriers for the application of (hot) melt extrusion, the polymers should have suitable properties such us: thermoplasticity, suitable glass transition temperature or melting point, thermostability at required processing temperature, no unexpected chemical interaction with active ingredients etc. In this context, polyvinyl alcohol (PVA) is an excellent compound, which is suitable for (hot) melt extrusion, as carrier for pharmaceutically active ingredients. Polyvinyl alcohol (PVA) is a synthetic water-soluble polymer that possesses excellent film-forming, adhesive, and emulsifying properties. It is prepared from polyvinyl acetate, where the functional acetate groups are either partially or completely hydrolyzed to alcohol functional groups. As the degree of hydrolysis increases, the solubility of the polymer in aqueous media increases, but also the crystallinity of the polymer increases. In addition to this, the glass transition temperature varies depending on its degree of hydrolysis.

During hot melt extrusion, mixtures of active ingredients, thermoplastic excipients, and other functional processing aids, are heated and softened or melted inside of an extruder and extruded through nozzles into different forms. The obtained extrudate can be cut down into small beads or milled into fine powder. The milled extrudate powder can be compressed together with other additional excipients for tableting, such as binders or disintegrants, to make the direct compression of tablet possible.

In this method, thermoplastic polymer PVA may be mixed with a pharmaceutical active substance (API) and optional inert excipients and further additives. The mixture is fed into rotating screws that convey the powder into a heated zone where shear forces are imparted into the mixture, compounding the materials until a molten mass is achieved. The extrudate with solid dispersed API can be milled into fine powder and directly compressed into tablets with other excipients, such as binders or disintegrants. The solubility of API can be improved in the final dosage form of tablet, hi this way, tablets can be produced with a “controlled release” characteristic. Depending on the various ingredients and their quantitative proportions in the compositions, formulations of compressed tablets based on PVA can be prepared with instant or sustained release kinetic of the active ingredient.

Here the term “controlled release” is understood to mean that a drug (API) is delivered from a tablet at a desired rate for a desired length of time. In other words, this means that the active ingredient, such as a drug, is released to its target environment in a controlled fashion, rather than immediately. “Sustained release kinetic” is a mechanism to dissolve a drug from tablets or capsules over time in order to be released slower and steadier into the bloodstream while having the advantage that the drug dose has to be taken at less frequent intervals than Immediate-release formulations of the same drug, for example the need of only one or two tablets per day.

A characteristic of sustained release is that it not only prolongs action but it attempts to maintain drug levels within the therapeutic window to avoid potentially hazardous peaks in drug concentration following administration and to maximize therapeutic efficiency.

On the other hand, it is understood to mean that formulations designed for “instant release” deliver the drug from a tablet or capsule immediately to the environment to induce its activity. A corresponding release profile is desired, for example, for formulations of agents for acute severe pain in order to achieve a rapid relief. The same applies to stomach remedies, which should act immediately in acute cases. In general “instant release” formulations provide the comprising API immediately to the environment within a very short time, so that an effective amount of the active ingredient is released after 30 minutes and the maximum concentration in the body fluid is reached after about 60 minutes.

Depending on the ingredients and the nature of the formulations, the release can also take place in a shorter period of time or slightly longer. However, it is essential for “instant release” formulations that their action generally lasts for a maximum of several hours and has to be re-dosed several times over the course of the day in order to achieve a lasting effect. In addition, “instant release” formulations are usually lower in dosage in order to avoid toxic situations, which can occur because of a fast and high release of API shortly after the administration of corresponding “instant release” tablets or capsules.

U.S. Pat. No. 5,456,923 A provides a process for producing a solid dispersion, which overcomes disadvantages of the conventional production technology for solid dispersions. The process comprises employing a twin-screw extruder in the production of a solid dispersion. In accordance with this, a solid dispersion can be expediently produced without heating a drug and a polymer up to or beyond their melting points and without using an organic solvent for dissolving both components and the resulting solid dispersion has excellent performance characteristics. The process claims a polymer that is natural or synthetic and can be employed as a raw material where the polymer's functions are not adversely affected by passage through the twin screw extruder.

EP 2 105 130 A1 describes a pharmaceutical formulation comprising a solid dispersion having an active substance embedded in a polymer in amorphous form, and an external polymer as a recrystallization inhibitor independently of the solid dispersion. The external polymer is claimed as a solution stabilizer. The active substance should be sparingly soluble or less sparingly soluble in water. Thermoplastic polymers are claimed as drug carriers to form a solid dispersion. It is claimed that the solid dispersion is obtained by melt extrusion. The process comprises melting and mixing the polymer and the active ingredient, cooling, grinding, mixing with the external polymer, and producing a pharmaceutical formulation. It is claimed that the melting is carried out at a temperature below the melting point of the drug. It is also claimed that the melting is carried out at a temperature above the T_{g} or melting point of the polymer, but from 0.1-5° C. below the melting point of the API. The melting point of pharmaceutical grades of PVA is normally above 178° C., although the glass transition temperature is in the range of 40-45° C.

### Problem to be Solved

Experiments have shown, that it is very difficult to mill extruded PVA into powders having fine particles, which in turn is an important condition for direct compression of PVA powders into tablets in order to obtain tablets having a satisfactory hardness and low friability.

In addition, the previous attempts have shown that there is a need for the addition of a certain amount of binder materials even if milled PVA powders have particles which seem to be fine enough for direct compression. This means, in general, additional binders in an amount of about 50% by weight of the tablet composition have to be added. But this limits the possible drug loading efficiency per tablet, because the drug has to be added in the form of a dispersion in a PVA matrix, wherein PVA as the functional polymer makes it possible to formulate crystalline APIs in the required amorphous state. Accordingly, it is desirable to be able to provide corresponding formulations which enable a higher active substance concentration in such compressed tablets.

Other problems refer to the disintegration characteristic of these tablets. As PVA is well known as very hydrophilic polymer forming a gel layer on surfaces of compressed tablets in aqueous medium, which blocks the disintegration of tablet. Corresponding tablets containing extruded dispersions of API and PVA are even more difficult to be disintegrated than the tablets without any API. The received drug containing tablet doesn't actually disintegrate.

The classical compounds for improving disintegration, such as VIVASTAR® (sodium starch glycolate) or croscarmellose sodium, have no effect on disintegration properties of PVA tablets. This means, that there is a need for new compositions to improve the disintegration of the tablets.

A further disadvantage of these PVA comprising tablets is that the gel layer on the surface of PVA tablet blocks the release of API, and may promote re-crystallization of API within the core of the tablets, because the API suffers a super saturated state inside of the tablet.

Usually the disintegration of a PVA dispersion based tablet is a very slow process and lasts for several hours and sometimes for more than 48 h. Therefore, it is desirable to provide various tablet compositions for the production of tablets based on milled PVA extrudate, having a “controlled release kinetic” of the comprising drug, for both tablet formulations with sustained release characteristics in an acceptable time as well as for those with an instant release characteristic.

### SUMMARY OF THE INVENTION

Surprisingly it was found by experiments that, for the direct compression of tablets, only if the extrudate with PVA and API is cryo-milled into powders having particles sizes ≤200 μm (d₅₀), preferably in the range of 60 to 120 μm (d₅₀), most preferred in the range of 70 to 110 μm (d₅₀), the direct compression is feasible. This milled extrudate powder shows good flowability, which eases the process of direct tableting. In particular, these improved properties are found for milled extrudate powders based on polyvinyl alcohol (PVA), having a particle size distribution of d₁₀=20±10 μm, d₂₀=40±10 μm, d₅₀=90±30 μm, d₉₀=200±30 μm, d₉₉=300±50 μm.

These particular polyvinyl alcohol grades fulfilling said conditions are preferably selected preferably from the group: PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-79, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88.

Accordingly, a PVA grade is subject matter of the present invention, which is suitable as thermoplastic polymer for HME and also suitable for one of the downstream formulation process of HME: direct tablet compression. In one embodiment of the invention polyvinyl alcohol as described above is extruded and milled homogeneously with at least one active pharmaceutical ingredient, whereby this milled powder is storage and transport-stable, and shows a suitable flowability for direct compression and which leads to an strong enough tablet hardness after compression. This powdery composition may comprise at least one additive selected from the group binder material, salt to reduce the cloud point of PVA, disintegrant, antioxidants, stabilizing agents, solubility-enhancing agents, pH control agents and flow regulators.

In a further embodiment of the invention the powdery composition of the present invention is a milled extrudate powder, comprising polyvinyl alcohol and optionally one or more further excipient(s) with particle sizes in the range of ≤200 μm (d50), preferably in the range of 60 to 120 μm (d50), most preferred in the range of 70 to 110 μm (d50). In particular, it is a milled powder comprising polyvinyl alcohol and optionally one or more further excipient(s) having a particle size distribution of d₁₀=20±10 μm, d₂₀=40±10 μm, d₅₀=90±30 μm, d₉₀=200±30 μm, d₉₉=300±50 μm.

Thus, the present invention also consists in a method for producing the extrudate powder according to the invention with improved properties for the directly compressed tablets. Said method or process for producing compressed tablets is characterized in that the extrudate of ingredients including polyvinyl alcohol and API as characterized above is processed in miller to a fine powder, and that then direct compressed into tablets for control released dissolution.

The particular advantage of the present invention is that the obtained milled extrudate powder can be directly compressed into tablets. Moreover, with additional excipients of tableting, the release kinetic of tablets can achieve not only instant but also sustained release of API, which overcomes the dissolution limitation of the compressed tablets based on PVA. The process according to the present invention includes the steps of
a) cryo-milling of extrudate from polyvinyl alcohol (PVA) and API to a powder having particle sizes in the range of ≤200 μm (D50), preferably in the range of 60 to 120 μm (D50), most preferred in the range of 70-110 μm (D50)
b) mixing this milled powder homogeneously with at least one active pharmaceutical ingredient, and optionally with at least one additive selected from the group binder material, disintegrant, pore builder surface active material, antioxidant, stabilizing agent, solubility-enhancing agents, pH control agents and flow regulators and
c) feeding this powdery composition evenly into the tablet compression machine and compressed them directly into tablets.

This process can be performed particularly well, if in step a) polyvinyl alcohol (PVA) based extrudate is milled to a powder having a particle size distribution of, d₁₀=20±10 μm, d₂₀=40±10 μm, d₅₀=90±30 μm, d₉₀=200±30 μm, d₉₉=300±50 μm namely when solid polyvinyl alcohol (PVA) having pharmaceutical grade is applied which is characterized having a viscosity ≤40 mPa·s, the viscosity being measured on 4% aqueous solution at 20° C. DIN 53015, is milled to a powder having a particle size distribution of d₁₀=20±10 μm, d₂₀=40±10 μm, d₅₀=90±30 μm, d₉₀=200±30 μm, d₉₉=300±50 μm. In this case very particularly preferred is the use of polyvinyl alcohol (PVA), selected from the group: PVA 2-98, PVA 3-80, PVA 3-83, PVA 3-85, PVA 3-88, PVA 3-98, PVA 4-85, PVA 4-88, PVA 4-98, PVA 5-74, PVA 5-82, PVA 5-88, PVA 6-88, PVA 6-98, PVA 8-88, PVA 10-98, PVA 13-88, PVA 15-99, PVA 18-88, PVA 20-98, PVA 23-88, PVA 26-80, PVA 26-88, PVA 28-99, PVA 30-75, PVA 30-92, PVA 30-98, PVA 32-80, PVA 32-88, PVA 40-88, most preferred from the group: PVA 3-88, PVA 4-88, PVA 5-74, PVA 5-88, PVA 8-88, and PVA 18-88, which is milled to a powder having a particle size distribution of d₁₀=20±10 μm, d₂₀=40±10 μm, d₅₀=90±30 μm, d₉₀=200±30 μm, d₉₉=300±50 μm.

Thus, a directly compressed tablet form from PVA extrudate, which is characterized as disclosed herein and which is obtainable by a process as characterized here, is the subject of the present invention. By making available this directly compressed tablet disadvantages as described above can be overcome in a simple manner.

### DETAILED DESCRIPTION OF THE INVENTION

The present invention relates to a downstream formulation process of hot melt extrusion: from extrudate to compressed tablet with improved micronized extrudate powder based on polyvinyl alcohol (PVA), and that due to its improved properties can be better directly compressed into tablets. Furthermore, this invention refers also to the compositions of compressed tablets which are able to deliver a controlled release (instant release and sustained release) kinetic of pharmaceutical ingredients comprising polyvinyl alcohol as carrier matrix and their use.

While the making and using of various embodiments of the present invention are discussed in detail below, it should be appreciated that the present invention provides more applicable inventive concepts than described here in detail. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “a homogenous melt, or mixture or form” refers to the various compositions that can be made by extruding the made-up source material, which is prepared by milling and combining selected sieve fractions.

As used herein, the term “heterogeneously homogeneous composite” refers to a material composition having at least two different materials that are evenly and uniformly distributed throughout the volume and which are prepared of the one or more APIs and the one or more pharmaceutically acceptable excipients, including a pretreated PVA into a composite.

As used herein, “bioavailability” is a term meaning the degree to which a drug becomes available to the target tissue after being administered to the body. Poor bioavailability is a significant problem encountered in the development of pharmaceutical compositions, particularly those containing an active ingredient that is not highly soluble.

As used herein, the phrase “pharmaceutically acceptable” refers to molecular entities, compositions, materials, excipients, carriers, and the like that do not produce an allergic or similar untoward reaction when administered to humans in general.

As used herein, “pharmaceutically acceptable carrier” or “pharmaceutically acceptable materials” includes any and all solvents, dispersion media, coatings, antibacterial and antifungal agents, isotonic and absorption delaying agents, and the like. The use of such media and agents for pharmaceutical active substances is well known in the art.

The API (active pharmaceutical ingredient) may be found in the form of one or more pharmaceutically acceptable salts, esters, derivatives, analogs, prodrugs, and solvates thereof. As used herein, a “pharmaceutically acceptable salt” is understood to mean a compound formed by the interaction of an acid and a base, the hydrogen atoms of the acid being replaced by the positive ion of the base.

As used herein, “poorly soluble” refers to having a solubility means the substance needs 100 ml solvent to dissolve 1 g substance.

A variety of administration routes are available for delivering the APIs to a patient in need. The particular route selected depends upon the particular drug selected, the weight and age of the patient, and the dosage required for therapeutic effect. The pharmaceutical compositions may conveniently be presented in unit dosage form. The APIs suitable for use in accordance with the present disclosure, and their pharmaceutically acceptable salts, derivatives, analogs, prodrugs, and solvates thereof, can be administered alone, but will generally be administered in admixture with a suitable pharmaceutical excipient, diluent, or carrier selected with regard to the intended route of administration and standard pharmaceutical practice.

The excipients and adjuvants that may be used in the presently disclosed compositions and composites, while potentially having some activity on their own, for example, antioxidants, are generally defined for this application as compounds that enhance the efficiency and/or efficacy of the effective ingredients. It is also possible to have more than one active ingredient in a given solution, so that the particles formed contain more than one active ingredient.

As stated, excipients and adjuvants may be used to enhance the efficacy and efficiency of the APIs dissolution.

Depending on the desired administration form the formulations can be designed to be suitable in different release models, which are well known to the skilled person, as there are: immediate, rapid or extended release, delayed release or for controlled release, slow release dosage form or mixed release, including two or more release profiles for one or more active pharmaceutical ingredients, timed release dosage form, targeted release dosage form, pulsatile release dosage form, or other release forms.

The resulting composites or compositions disclosed herein may also be formulated to exhibit enhanced dissolution rate of a formulated poorly water soluble drug.

The United States Pharmacopeia-National Formulary mandates that an acceptable polyvinyl alcohol for use in pharmaceutical dosage forms must have a percentage of hydrolysis between 85 and 89%, as well as a degree of polymerization between 500 and 5000. The degree of polymerization (DM) is calculated by the equation:

DM=(Molar Mass)/((86)−(0.42(the degree of hydrolysis)))

The European Pharmacopoeia mandates that an acceptable polyvinyl alcohol for use in pharmaceutical dosage forms must have an ester value no greater than 280 and a mean relative molecular mass between 20,000 and 150,000. The percentage of hydrolysis (H) can be calculated from the following equation:

H=((100−(0.1535)(EV))/(100−(0.0749)(EV)))×100

Where EV is the ester value of the polymer. Thus, only polymers with a percentage of hydrolysis greater than 72.2% are acceptable according to the European Pharmacopoeia monograph.

As already mentioned above, commercially available polyvinyl alcohols in particulate form have poor flow behavior, especially if they are characterized by low viscosities (measured in a 4% aqueous solution at 20° C.). Accordingly, these powders have no continuous trouble-free flow. However, the latter is a prerequisite for a uniform feed to the processing of such powder materials.

Theoretically, powders, whose particle shapes are rather round and spherical, in general have the best flow behavior. Accordingly, in the past, attempts have been made to produce polyvinyl alcohol powders already directly by its synthesis with spherical particles. For example, from DE 38 11 201A a method is known for producing of spherical particles by suspension polymerization. However, this reaction requires a special adjustment of the reaction conditions. In addition, this reaction has to be followed by a hydrolysis reaction. With different particle sizes, it is difficult to achieve a uniform degree of hydrolysis of the polymer particles. By this method, polyvinyl alcohol powders are produced having viscosities of 80 mPa·s or higher.

Therefore, for the production of polyvinyl alcohol powders, which are comparable with those of the present invention, this method provides no alternative, especially as here PVA grades are desirable having viscosities of ≤40 mPa·s.

Now, it was found that polyvinyl alcohol grades having viscosities of 40 mPa·s are also suitable to be manufactured by melt extrusion, if they are pretreated as disclosed in the following and a homogenously dispersed solid solution of pharmaceutical active ingredient in polyvinyl alcohol can be produced by extrusion and the received drug containing PVA powder can be fed without problems into the feeder.

In this way also poorly soluble pharmaceutical active ingredients (from BCS class II and IV) can be homogeneously mixed with PVA to build a solid dispersion. Furthermore, it was found by experiments that PVA in the different degrees of hydrolysis having viscosities of ≤40 mPa·s can be homogeneously mixed by melt extrusion with poorly soluble active ingredients, especially with PVA that is in accordance with the European Pharmacopoeia monograph and which is a pharmaceutically acceptable PVA with hydrolysis grades greater than 72.2%, and especially which includes grades of PVA that are pharmaceutically acceptable by either the USP (hydrolysis between 85-89%) or Ph. Eur. (hydrolysis grades greater than 72.2%). These PVA qualities have a molecular weight in the range of 14,000 g/mol to 250,000 g/mol.

Micronized compositions according to the invention may comprise at least a biologically active ingredient combined with a PVA that is pharmaceutically acceptable, which is combined with another pharmaceutically acceptable polymer. Such pharmaceutically acceptable polymer can also be selected from the group of hydrophilic polymers and can be a primary or secondary polymeric carrier that can be included in the composition disclosed herein and including polyethylene-polypropylene glycol (e.g. POLOXAMER™), carbomer, polycarbophil, or chitosan, provided that they are as free-flowing powder and are extrudable polymers. Hydrophilic polymers for use with the present invention may also include one or more of hydroxypropyl methylcellulose, carboxymethylcellulose, hydroxypropyl cellulose, hydroxyethyl cellulose, methylcellulose, natural gums such as gum guar, gum acacia, gum tragacanth, or gum xanthan, and povidone. Hydrophilic polymers also include polyethylene oxide, sodium carboxymethycellulose, hydroxyethyl methyl cellulose, hydroxymethyl cellulose, carboxypolymethylene, polyethylene glycol, alginic acid, gelatin, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

In general, it must be considered that there are special requirements for polymers used as hot melt extrusion excipients:

The polymer must be thermoplastic, must have a suitable glass transition temperature and a high thermal stability. The polymer must have no toxic properties and must have a high biocompatibility, etc. Therefore, pharmaceutical grades of polyvinyl alcohol (PVA), which are chosen here for the preparation of formulations comprising active ingredients by hot melt extrusion, are those having a low viscosity.

Moreover, for one of the downstream formulations of hot melt extrusion, preferably a direct compressed tablet, the extrudate should be milled into fine powder with suitable particle size and size distribution, in order to make the feeding and direct compression feasible and in order to obtain tablets, which can deliver a desired controlled release kinetic, especially instant or sustained release.

Polyvinyl alcohol (PVA) is a synthetic polymer, which is produced by polymerization of vinyl acetate and partial hydrolysis of the resulting esterified polymer. As already mentioned above, chemical and physical properties of polyvinyl alcohol, such as viscosity, solubility, thermal properties, etc. are very depending on its degree of polymerization, chain length of PVA polymer, and the degree of hydrolysis.

PVA can be used for the production of different formulations for various modes of administration to treat a variety of disorders. Accordingly, PVA is processed in a wide range of pharmaceutical dosage forms, including ophthalmic, transdermal, topical, and especially, oral application forms.

As mentioned above, it is for the successful industrial processing of a solid dosage form, including the steps

1) an extrusion process
2) a milling process
3) a direct compression process into tablet,
necessary that a uniform continuous metering is possible in the extruder, miller and tablet compression machine.

Now it was found by experiments, that for direct compression, the milled extrudate must have suitable particle characteristics, including appropriate particle sizes, and flowability or fluidity. It was also found, that extruded and milled polyvinyl alcohol powder of pharmaceutical grade as characterized above and having particle sizes in the range of 200 μm (d₅₀), preferably in the range of 60 to 120 μm (d₅₀), most preferred in the range of 70-110 μm (d₅₀) show improved feasibility of direct compression.

In particular, these powders exhibit improved feasibility of direct compression, when the particle size distribution is in the range of d₁₀=20±10 μm, d₂₀=40±10 μm, d₅₀=90±30 μm, d₉₀=200±30 μm, d₉₉=300±50 μm, namely when solid polyvinyl alcohol (PVA) having pharmaceutical grade is applied, which is characterized having a viscosity ≤40 mPa·s, the viscosity being measured on 4% aqueous solution at 20° C. DIN 53015. In this case very particularly preferred is the use of polyvinyl alcohol (PVA) having pharmaceutical grade, selected from the group: PVA 2-98, PVA 3-80, PVA 3-83, PVA 3-85, PVA 3-88, PVA 3-98, PVA 4-85 PVA 4-88, PVA 4-98, PVA 5-74, PVA 5-82, PVA 5-88, PVA 6-88, PVA 6-98, PVA 8-88, PVA 10-98, PVA 13-88, PVA 15-99, PVA 18-88, PVA 20-98, PVA 23-88, PVA 26-80, PVA 26-88, PVA 28-99, PVA 30-75, PVA 30-92, PVA 30-98, PVA 32-88, PVA 40-88, most preferred from the group: PVA 3-88, PVA 4-88, PVA 5-74, PVA 5-88, PVA 8-88, and PVA 18-88, which is extruded with API and further milled to a powder having a particle size distribution of d₁₀=20±10 μm, d₂₀=40±10 μm, d₅₀=90±30 μm, d₉₀=200±30 μm, d₉₉=300±50 μm.

The milled extrudate powders, comprising particles larger than in the range of about 200 μm (d₅₀), cannot be compressed into tablets, which are hard enough, not even with additional binder materials.

It was also found by experiments, that 0%-15% by weight of binder material is needed, but not limited with 0%-15%, to improve the hardness and friability of the compressed tablet. The binder materials in the case of PVA extrudate can also be added in an amount of up to 50% to make the direct compression feasible.

It is well known that the gel layer on the surface of PVA tablet blocked the release of API, and may promote recrystallization of API within the tablets, because the API suffers a super saturated state inside of the tablet. The classic disintegrants such us VIVASTAR® (sodium starch glycolate) or crosscarmellose sodium had no effect on disintegration property to PVA tablet. The tablet based on PVA disintegrate normally very slowly for several hours and so that they deliver a super sustained dissolution release kinetic

Surprisingly, tablet compositions can be provided solving the problem described above:

1. Based on milled PVA/API extrudate (about 50-85% extrudate within the tablet, which make the high API loading of tablet possible).

Contained at least binder material (microcrystalline cellulose for example) as binder 0-15% to achieve an excellent hardness or strength of the tablets. But the amount of binder material is not limited with 0%-15%. In the case of PVA, up to 50% binder material can be added to make the direct compression feasible.

2. Contained inorganic salt (e.g. KHCO₃ or NaCl) to reduce the cloud point of PVA within the tablet, in order to break the hydro gel layer of PVA and make disintegration of the tablets possible 0-30%.
4. Contained pore builder (e.g. lactose) 0-30%.
5. Contained disintegrate regulator (e.g. Kollidone® CL-F, Croscarmallose sodium, Polyplasdon® XL-10) as 0-15%.

The new tablet compositions make the disintegration of the tablets based on extrudate PVA powder from impossible to possible, can protect the API against recrystallization and deliver a control released (instant release and sustained release) kinetic of API.

### EXAMPLES

Even without any further explanations, it is assumed that a person skilled in the art can make use of the above description in its widest scope. The preferred embodiments and examples are therefore to be regarded merely as descriptive but in no way limiting disclosures.

For better understanding and for illustration, examples are given below which are within the scope of protection of the present invention. These examples also serve for the illustration of possible variants.

The complete disclosure of all applications, patents and publications mentioned above and below are incorporated by reference in the present application and shall serve in cases of doubt for clarification.

It goes without saying that, both in the examples given and also in the remainder of the description, the quoted percentage data of the components present in the compositions always add up to a total of 100% and not more. Given temperatures are measured in ° C.

Now, in order to carry out the following experiments, extrudate with PVA and API was cryo-milled into three charges under different milling conditions (definition of method is following) to obtain different particle sizes and particle distributions of extrudate powders:

Charge 1: Particle size in the range of 100 μm (d50)
Charge 2: Particle size in the range of about 200 μm (d50)
Charge 3: Particle size in the range of 350 μm (d50)

Before milling, PVA was physically blended with active ingredients in an amount of 20-60% by weight, with or without additional plasticizers. The mixture was extruded under suitable conditions (depends on API) and cryo-milled into fine powder, which is characterized regarding to the flowability, homogeneity and feasibility of direct compression into tablets.

The analysis of the data obtained indicated, that cryo-milled PVA powder with particles having an average particle size of ≤100 μm and a particle distribution of:

| | Dv5 | Dv10 | Dv20 | Dv25 | Dv30 | Dv50 | Dv75 | Dv90 | Dv95 |
|---|---|---|---|---|---|---|---|---|---|
| | (μm) | (μm) | (μm) | (μm) | (μm) | (μm) | (μm) | (μm) | (μm) |
| Group A | 13.176 | 21.06 | 37.76 | 46.76 | 55.81 | 92.87 | 152.83 | 219.32 | 262.04 |

are the most suitable powders to be compressed into tablets. The blended mixture with other excipients such as binder materials or disintegrants was also homogenous and had good flowability to be feeded in the tableting machine. Extrudate powder larger than 200 μm (d50) was difficult to be compressed into tablets, which was hard enough and the homogeneity of the tablet was also a problem.

Methods and Materials

1. Raw Materials and Manufacturing Method

1.1 Materials

Raw Material:
Poly vinyl alcohol 4-88, excipient EMPROVE® exp Ph Eur, USP, JPE, Article No. 1.41350, Merck KGaA, Darmstadt, Germany
Indomethacin, active ingredient, Sigma, 17378-100G
Itraconazole, active ingredient, Selectchemie, AG, Germany
Microcrystalline cellulose (MCC), VIVAPUR®102 Premium, Ph. Eur., NF, JP, JRS Pharma Rosenberg, Germany
Magnesium stearate, Parteck® LUB MST, EMPROVE exp Ph Eur, BP, JP, NF, FCC 1.00663, Merck KGaA, Darmstadt, Germany
Lactose (Ludipress®), BASF, Ludwigshafen, Germany
Siliciumdioxide, EMPROVE exp, Nr. 1.13126 Merck KGaA, Darmstadt, Germany
KHCO₃, Merck KGaA, Darmstadt, Germany
NaCl, Merck KGaA, Darmstadt, Germany
Kollidone® CL-F, BASF, Ludwigshafen, Germany

1.2 Experiments & Characterization Methods

1.2.1 Extrusion Process

Equipment:
Physical blend of composition for hot melt extrusion, including active ingredients: TURBULA® Shaker-Mixer
Brabender® Mini-Compounder (KETSE 12/36 D)
Brabender® Pelletizer
The mixture of PVA and active ingredient were blended using TURBULA® Shaker-Mixer homogeneously (the concentration of polymer and active ingredient depends on the types and physical properties of them). The mixture was then loaded into the extruder with well designed extrusion parameters, such as feeding rate, screw design, screw speed, extrusion temperature etc. The set up of those parameters depend also on the types and physical properties of polymer and active ingredients. The extrudate was cut into 1-3 mm small beads with Brabender® Pelletizer.

1.2.2 Milling Process
Equipment in lab: Ultra-Zentrifugalmühle ZM 200 200-240V, 50/60 Hz
Scale up equipment: Mill equipment for extrudate milling: aeroplex spiral jet mill, type 200 AS Hosokawa Alpine, Augsburg, Germany

Milling conditions: with liquid nitrogen as cold grinding. The desired particle sizes are produced empirically in particular by varying the grinding temperature, to control the particle size of PVA. The grinding conditions are varied until the desired particle size is obtained.

TABLE 1
cryo-milling methods for 3 group

| Group | Sieve Type | Rotation speed |
|---|---|---|
| A | 0.35 mm | 18000 rpm |
| B | 1.00 mm | 18000 rpm |
| C | 1.00 mm | 10000 rpm |

Goal of particle size & distribution of each group:

Group A: Extrudate Particle Size→≤100 μm (d50)
Group B: Extrudate Particle Size→about 200 μm (d50)
Group C: Extrudate Particle Size→about 350 μm (d50)

Particle Size & Distribution Analysis

Particle size determination is carried out by laser diffraction with dry dispersion: Mastersizer 2000 with dispersing Scirocco 2000 (Malvern Instruments Ltd. UK.), Provisions at 1, 2 and 3 bar backpressure; Evaluation Fraunhofer; Dispersant RI: 1000, obscuration limits: 0.1-10.0%, Tray Type: General Purpose, Background Time: 7500 msec Measurement Time: 7500 msec, implementation in accordance with ISO 13320-1 and the details of the technical manual and the specifications of the equipment manufacturer; Information in Vol-%.

Angle of Repose (DIN ISO 4324)

The Angle of repose gives information about the flowability of the milled extrudate for example in the tablet compression machine. First of all you have to adjust the disk (with the stand on it). To set up the equipment, proceed as the picture. After that you can fill in your powder into the glass funnel (two-thirds).

Attention: Ensure that the flap under the funnel is closed!

Now you can start opening the flap and let your powder trickle into the transparent plastic receptacle under the glass funnel. If necessary, use the stirrer! When the powder is on the wraparound edge of the plastic receptacle, close the flap and measure the height of the cone. Repeat it five-times.

Mathematical formula for tamped density:

$$Arc tan ( 2 ⋆ \frac{height}{diameter} )$$

1.2.3 Direct Compression Process

Equipment in lab: a hand tablet press (Fa. Röltgen). The tablets have different sizes:
500 mg tablets→ϕ011 mm punch, round, flat, facet
1000 mg tablets→ϕ015 mm punch, round, flat, facet, engraving
The tested press forces were from 5 kN up to 30 kN

Scale up equipment: Romaco Kilian (STYL'ONE; Type: Evolution):
1000 mg oblong tablets, ϕ19 mm punch, engraving
The tested press forces were from 5 kN up to 40 kN

Tablethardness, -Average, -Weight and Tablet Weight:
For smaller batches (5 tablets), the tablethardness is tested on “Tablet Tester 8M Dr. Schleuniger, Pharmatron”. The measurements are made at the day of process (in-process control) and one day after production. (Balance of Erweka Multicheck 5.1: Sartorius CPA 64)
For scale-up tests 20 tablets was tested on “Erweka Multicheck 5.1 (Fa. Erweka, Germany)”.

1.2.4 Dissolution

For the real time dissolution performance, we used following equipments:

System 1:
Sotax AT 7 on/offline
Pumpe CY-7-50
Fraktionssammler: C613 14 Kanal 3 Wege Ventilbalken für Reagenzgläser
Agilent 8453 Photometer

System 2
Sotax AT 7 on/offline
Pumpe CP 7-35
Fraktionssammler: C 613 14 Kanal 3 Wege Ventilbalken für Vials
Photometer Analytik Jena Specord 200 plus

2. Results

2.1 Particle Size and Distribution

A milled extrudate powder having this particle size distribution is characterized by the logarithmic plot of particle sizes ranging up to 100 microns to their volume percentage:

TABLE 2
particle size & distribution of milled extrudate with 30% itraconazole and 70% PVA

| | Dv5 | Dv10 | Dv20 | Dv25 | Dv30 | Dv50 | Dv75 | Dv90 | Dv95 |
|---|---|---|---|---|---|---|---|---|---|
| | (μm) | (μm) | (μm) | (μm) | (μm) | (μm) | (μm) | (μm) | (μm) |
| Group A | 13.176 | 21.06 | 37.76 | 46.76 | 55.81 | 92.87 | 152.83 | 219.32 | 262.04 |
| Group B | 20.77 | 34.32 | 64.74 | 81.32 | 98.43 | 172.15 | 295.64 | 430.17 | 514.75 |
| Group C | 37.76 | 65.46 | 137.07 | 176.54 | 213.17 | 342.98 | 527.58 | 712.4 | 823.04 |

2.2 Flowability

There are differences in the flowability, if extrudate powders as characterized above (Group A, Group B and Group C) are compared with each other and there are additional effects in the flowability if the different extrudate powders are mixed with APIs (Active Pharmaceutical Ingredients), so that flowabilities differ between mixtures with and without APIs.

2.3 Feasibility of Direct Compression

2.3.1 Relationship Between Particle Properties and Tablet Hardness

With this compression experiment, we found that the milled extrudate with d₅₀ 100 μm (group A) can be easily compressed in tablets, which is hard enough: under 10 KN compression force can achieve 125 N hardness and under 20 KN compression force can achieve 290 N hardness. If the milled extrudate particle larger than 200 μm (d₅₀), it can also be compressed into tablets but the hardness of tablets is not strong enough.

TABLE 3
tablets properties prepared from powders with different particle size
and distribution:

| Particle | | | | |
|---|---|---|---|---|
| size & | | | | |
| Distri- | | | | |
| bution | 10 KN compression force | | 20 KN compression force | |
| of milled | Hardness | Tablet | Hardness | Tablet |
| extrudate* | (N) | Weight (g) | (N) | Weight (g) |
| Group A | 124 ± 7.45 | 0.993 ± 0.015 | 288 ± 2.69 | 0.980 ± 0.011 |
| Group B | 83 ± 2.78 | 0.994 ± 0.010 | 213 ± 7.05 | 0.978 ± 0.030 |
| Group C | 57 ± 3.56 | 1.063 ± 0.015 | 162 ± 6.02 | 1.027 ± 0.019 |

(*Tablet properties depend on the tablet form and the composition of tablet, even with the same type and amount of milled extrudate. The composition of model tested tablets in this table: 15 mm, round form; 50% milled extrudate, 10% microcrystalline cellulose, 16% NaCl, 17.5% lactose, 0.5% magnesium stearate, 1.0% silicium dioxide and 5% Polyplasdone XL)

2.3.2 Relationship Between Binder Material Concentration and Tablet Hardness

We evaluated that in the case of extrudate based on PVA, the hardness of extrudate will be improved with the increasing of MCC concentration till 15%. If MCC increases to more than 15% (20% e.g.), there will be no improvement of tablet hardness or even worse than 15% MCC.

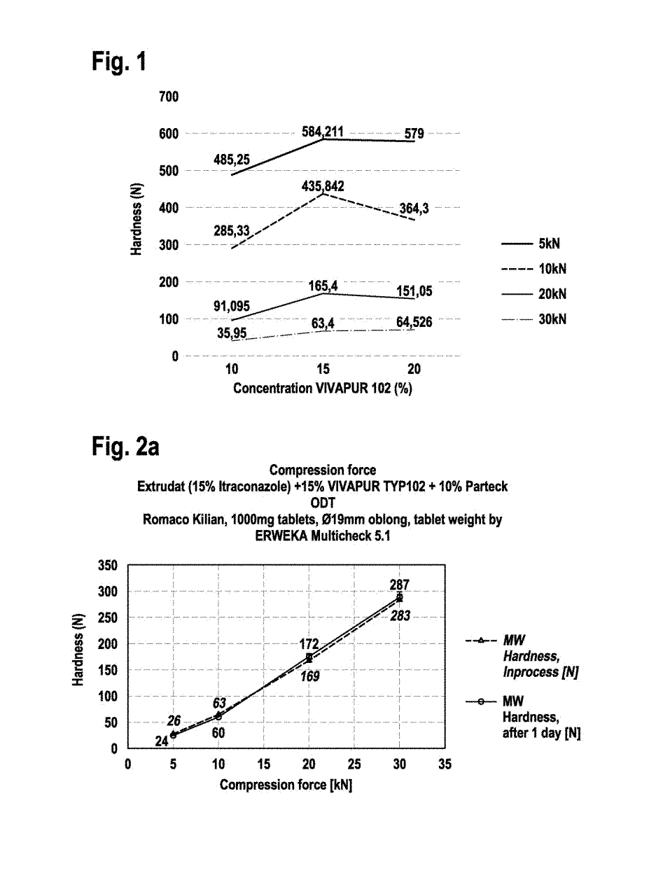

FIG. 1: Tablet strength of milled powder group A (with 30% API, d50=100 μm), tablet form: 19 mm/oblong; tablet composition: with different concentrations of MCC (VIVAPUR TYPE102), the rest is milled extrudate.

2.3.3 Relationship Between Compression Force and Tablet Hardness

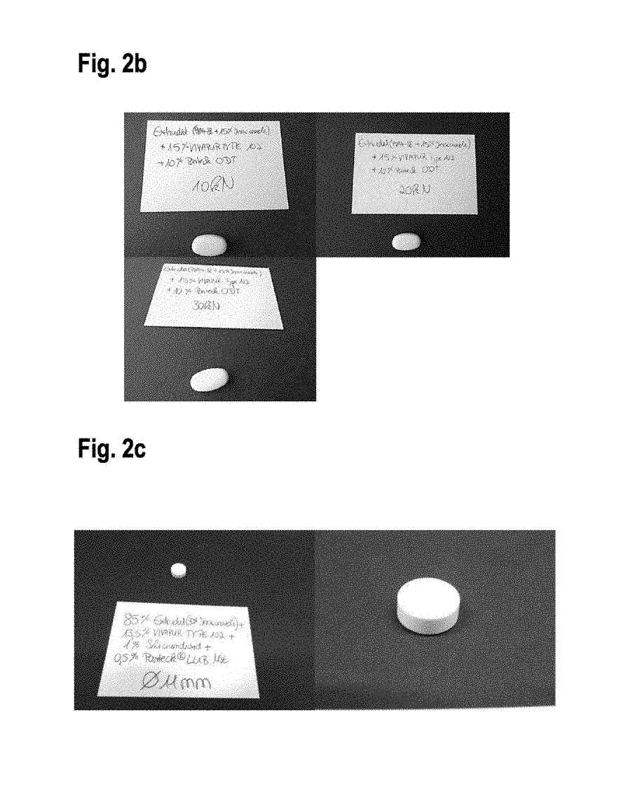

FIG. 2a: relationship between compression force and tablet handness (Tablet composition: 75% extrudate powder group A, 15% binder material, 10% pore builder) (Hardness [kN] versus compression force [kN])

FIG. 2b: Photo (1): 19 mm/oblong tablets

2.3.4 Relationship Between Tablet Form and Tablet Hardness

TABLE 4
influence of compression force and tablet diameter on the tablet
hardness (tablet composition: 85% extrudate powder from group A, 13.5%
VIVAPUR TYPE 102, 1% SiO2, 0.5% Parteck LUB Mst)

| Diameter of | Strength | Strength | Strength |
|---|---|---|---|
| tablet | (10 KN) | (20 KN) | (30 KN) |
| 11 mm/round | 302 ± 4 N | 461 ± 17 N | 506 ± 25 N |
| 15 mm/round | 226 ± 13 N | 411 ± 16 N | 527 ± 21 N |

FIG. 2c: shows a Photo (2) of corresponding 11 mm/round tablets as disclosed in table 4

2.4 Dissolution of Compressed Tablets with Model API

2.4.1 Sustained Release Tablets

### Composition Example 1

TABLE 5
tablets composition 1 for sustained release
1000 mg sustained release tablet containing 125 mg itraconazole
(15 mm round/hardness 411 ± 16 N under the compressed force of
20 KN)

| compound | [mg] | % (w/w) |
|---|---|---|
| Milled extrudate group | 850 | 85 |
| A with 30% | | |
| itraconazole | | |
| VIVAPUR TYPE 102 | 135 | 13.5 |
| (MCC) | | |
| Silicon dioxide | 10 | 1 |
| Parteck ® LUB MST | 5 | 0.5 |
| (magnesium stearate) | | |

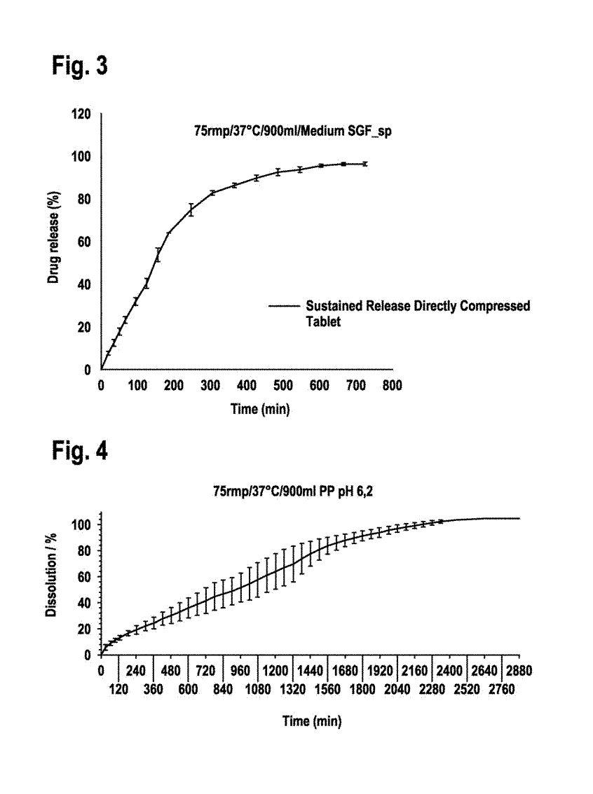

FIG. 3: sustained release of itraconazole tablet (Drug release (%) versus time (min))

### Composition Example 2

TABLE 6
tablets composition 2 for sustained release
328 mg sustained release tablet contained 85 mg in (10 mm round/
hardness 299 ± 0.71 N under the compressed force of 20KN)

| compound | [mg] | % (w/w) |
|---|---|---|
| Milled extrudate group A | 280.5 | 85 |
| with 30% indomethacin | | |
| VIVAPUR TYPE 102 | 44.55 | 14 |
| (MCC) | | |
| Silicon dioxide | 3.3 | 1% |

FIG. 4: sustained release of indomethacin tablet (Dissolution % versus time (min))

2.4.2 Instant Release Tablets

### Composition Example 1 (without MCC)

TABLE 7
tablets composition 1 for instant release
1000 mg instant release tablet contained 150 mg itraconazole
(10 mm round/130 N ± 6 N hardness under the compressed force
of 10 KN)

| compound | [mg] | % (w/w) |
|---|---|---|
| Milled extrudate group | 500 | 50 |
| A with 30% | | |
| itraconazole | | |
| Lactose | 300 | 30 |
| NaCl | 200 | 20 |

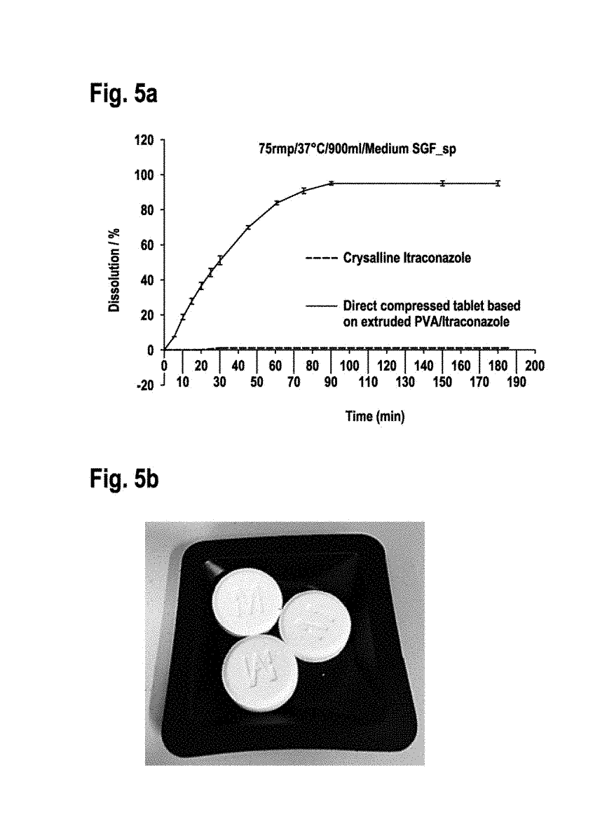

FIG. 5a: shows the Dissolution of instant release tablets with 50% PVA/API extrudate (without MCC)

FIG. 5b: shows a photo (3) of compressed tablets based on PVA and itaconazole extrudate.

### Composition Example 2

TABLE 8
tablets composition 2 for instant release
1000 mg instant release tablet containing 150 mg itraconazole
(10 mm round/264 N ± 9.8 N hardness under the compressed force
of 10 KN)

| compound | [mg] | % (w/w) |
|---|---|---|
| Milled extrudate group A | 500 | 50 |
| with 30% itraconazole | | |
| Lactose | 175 | 17.5 |
| NaCl | 160 | 16 |
| VIVAPUR TYPE 102 | 100 | 10 |
| (MCC) | | |
| Silicon dioxide | 10 | 1 |
| Parteck ® LUB MST | 5 | 0.5 |
| (magnesium stearate) | | |
| Polyplasdon XL | 50 | 5 |

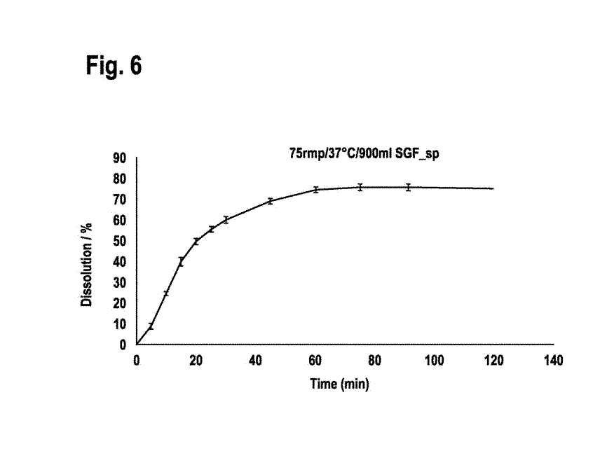

FIG. 6: shows the dissolution of instant release tablet with 50% PVA/API extrudate (with MCC)

2.5 Summary

Advantages of investigated powders and compositions:

1. The method to mill the extruded PVA/API into best particle size and distribution.
2. The benefit of the best particle size and distribution of milled PVA/API extrudate: excellent flowability and feasibility of direct tablet compression and excellent tablet hardness
3. Defined best microcrystalline cellulose (MCC) type with optimized particle size & distribution (d₅₀ around 100 μm) is the best binder material for milled extrudate based on PVA
4. The best concentration of MCC to improve the tablet hardness
5. Controlled release dissolution kinetic of final tablet can be achieved.

1. Polyvinyl alcohol (PVA) comprising powder, characterized in that it shows improved flowability and feasibility in direct compression to tablets after extrusion and milling to particle sizes in the range of ≤200 μm (d50), preferably in the range of 60 to 120 μm (d50), most preferred in the range of 70 to 110 μm (d50).

2. Polyvinyl alcohol (PVA) comprising powder according to claim 1, characterized in that it is milled after extrusion to a particle size distribution of d₁₀=20±10 μm, d₂₀=40±10 μm, d₅₀=90±30 μm, d₉₀=200±30 μm, d₉₉=300±50 μm.

3. Polyvinyl alcohol comprising powder according to claim 1, which is hot melt extruded or melt extruded before milling.

4. Polyvinyl alcohol comprising powder according to claim 1, characterized in having a viscosity ≤40 mPa·s in aqueous solution, the viscosity being measured on 4% w/v aqueous solution at 20° C. DIN 53015.

5. Polyvinyl alcohol comprising powder according to claim 1, which is selected from the group PVA 3-88, PVA 4-88, PVA 5-74, PVA 5-88, PVA 8-88, and PVA 18-88.

6. Polyvinyl alcohol comprising powder according to claim 1, characterized in that it shows improved flowability and feasibility in direct compression to tablets after extrusion and milling, thereby avoiding blocking during feeding of the powdery premix during the tableting process and allowing to carry out an uninterrupted process.

7. A powdery composition for the preparation of tablet formulations, comprising
a) polyvinyl alcohol powder according to claim 1 as carrier, which is an extruded and homogeneously milled powder,
b) at least one active pharmaceutical ingredient (API), and
c) optionally further additives
whereby this milled powder is storage and transport-stable.

8. A powdery composition according to claim 7, comprising at least one additive selected from the group of binder material, disintegrant, pore builder, surface active material, antioxidant, stabilizing agent, solubility-enhancing agents, pH control agents and flow regulators.

9. A powdery composition according to claim 7, comprising at least one additive selected from the group of binder material, salt for the reduction of the cloud point of PVA, disintegrant, pore builder, surface active material, antioxidant, stabilizing agent, solubility-enhancing agents, pH control agents and flow regulators.

10. A powdery composition according to claim 7, which is a pharmaceutical grade powder comprising polyvinyl alcohol, at least one active pharmaceutical ingredient (API) and optionally one or more further excipient(s) with particle sizes in the range of ≤200 μm (d50), preferably in the range of 60 to 120 μm (d50), most preferred in the range of 70 to 110 μm (d50).

11. A process for producing a solid pharmaceutical dosage form, characterized in that the powdery composition according to claim 8 is processed in a tableting machine into a compressed tablet.

12. A process according to claim 11, characterized in that the powdery composition is continuously and evenly fed into the tableting machine where it is processed into a homogeneous and hard tablet.

13. A process for producing a solid pharmaceutical dosage form according to claim 11, characterized in that
a) polyvinyl alcohol (PVA) having pharmaceutical grade is extruded with at least one active pharmaceutical ingredient and milled to a powder having particle in the range of ≤200 μm (d50), preferably in the range of 60 to 120 μm (d50), most preferred in the range of 70 to 110 μm (d50), and
b) that this powder is homogeneously mixed with at least one additive selected from the group of binder materials, salt to reduce the cloud point of PVA, disintegrant, pore builder, surface active material, antioxidant, stabilizing agent, solubility-enhancing agents, pH control agents and flow regulators and
c) that this powdery composition is evenly fed into the direct compression tableting machine by processing to a homogeneous and hard tablets.

14. A process according to claim 11, characterized in that in a first step polyvinyl alcohol (PVA) having pharmaceutical grade is milled to a powder having a particle size distribution of d₁₀=20±10 μm, d₂₀=40±10 μm, d₅₀=90±30 μm, d₉₀=200±30 μm, d₉₉=300±50 μm.

15. A process according to claim 10, characterized in that polyvinyl alcohol (PVA) having pharmaceutical grade, selected from the group PVA 3-88, PVA 4-88, PVA 5-74, PVA 5-88, PVA 8-88, and PVA 18-88, is milled to a powder having a particle size distribution of d₁₀=20±10 μm, d₂₀=40±10 μm, d₅₀=90±30 μm, d₉₀=200±30 μm, d₉₉=300±50 μm.

16. Direct compressed tablets form obtainable by a process according to claim 11.

17. Tablet composition according to claim 1 having controlled released kinetic.

18. Tablet composition according to claim 9 showing instant release of the comprising the API.

19. Tablet composition according to claim 1 showing sustained release of the comprising the API.
